(19) United States
(12) Patent Application Publication
(10) Pub. No.: US 2006/0292549 A1
(43) Pub. Date: Dec. 28, 2006

(54) Psychotropic drug screening device based on long-term photoconductive stimulation of neurons

(75) Inventor: Michael A. Colicos, Calgary (CA)

Correspondence Address: FISH & RICHARDSON P.C., PO BOX 1022, MINNEAPOLIS, MN, 55440-1022, US

(73) Assignee: NEUROSILICON, Calgary (CA)

(21) Appl. No.: 11/455,222

(22) Filed: Jun. 15, 2006

Related U.S. Application Data
(63) Continuation of application No. 60/691,012, filed on Jun. 15, 2005.

(51) Int. Cl. C12Q 1/00; G06F 19/00; C12M 3/00

(52) U.S. Cl. 435/4; 435/287.2; 702/19

(57) ABSTRACT

The invention pertains generally to the field of psychotropic drug discovery and neuroscience. More specifically, the invention refers to a device that allows the long-term stimulation of cultured neurons grown on a silicon die, thereby replacing the use of intact animal models. The device consists of a controlled sterile environment in which the neuronal cultures can grow, and into which specific candidate compounds (e.g., psychotropic drugs) are added. During the period of growth, which can be extended for months, specifically tailored patterned activity can be applied to the neuronal network, simulating the activity normally found in a brain. The neuronal networks are grown on a silicon surface which is targeted by a light source, which functions to alter the connectivity of the surface below a specific group of cells. Cells on this targeted region are caused to fire on a brief electrical pulse. Upon completion of the growth period the silicon die can be removed and subject to electrophysiological and biochemical analysis.

### CROSS REFERENCE TO RELATED APPLICATIONS

This application claims the benefit of priority under 35 U.S.C. §119 to U.S. provisional application Ser. No. 60/691,012, filed Jun. 15, 2005, the disclosure of which is incorporated herein by reference in its entirety.

The disclosures of U.S. provisional applications having Ser. Nos. 60/691,322, filed Jun. 15, 2005, and 60/699,829, filed Jul. 15, 2005, and U.S. utility application filed May 22, 2006 having Ser. No. 11/439,377 and identified by attorney docket no. 19040-003001, are also incorporated herein by reference in their entirety.

### FIELD OF THE INVENTION

The present invention pertains generally to the fields of drug discovery and neuroscience, and more particularly to methods and devices for automated screening of psychotropic drug candidates, based on their effect on cells stimulated over a long term, without the use of intact animal models.

### BACKGROUND

Psychotropic drugs are one of the leading areas of drug development. For example, selective serotonin reuptake inhibitors, or SSRIs, such as Paxil and its pharmaceutical cousins Prozac and Zoloft, are taken by millions of patients each year.

A psychotropic substance is a chemical that alters brain function, resulting in temporary changes in perception, mood, consciousness, or behavior. Psychotropic substances typically alter neuronal activity by changing one or more parts of the process of neuronal communication, though their specific modes of action are varied. Subtle local alterations of neurons have global effects that can result in changes to mood, perception or behavior. The term psychotropic is frequently used interchangeably with psychoactive, and both words refer to substances that alter neurons in a way that results in changes on a microscopic level, a macroscopic level, or both.

One characteristic of many psychotropic substances is that they can have a long-term effect on patterns of neuronal growth. For example, prolonged stimulation of a network of neurons in the presence of a psychotropic substance may lead to a different change in the characteristics of the network from comparable stimulation without the psychotropic substance being present. It has become important to understand such effects, both to understand the behavior of specific substances and to search for others having desirable effects. Currently, the only effective way to test the long-term effects of psychotropic drugs or their inhibitors on a mammalianbrain is to administer the drugs to test animals such as rats for long periods of time, extract brain tissue (e.g., by removing their brains), and perform tests on the extracted tissue. The tests may detect changes in the connectivity between the neurons or may study the altered biochemistry of the synapses, or, in some cases, the axons. This is too complicated an undertaking to be a routine laboratory endeavor, and there is usually little way to generate appropriate experimental controls for comparison. Improving the ability to monitor long-term effects of psychotropic drugs on neurons is of immediate and high importance because of the numbers of people who are dependent on such drugs. Nevertheless, methods of studying long-term effects on neuronal networks are not routine because culturing neurons for extended periods of time ex vivo has proved to be difficult.

Evaluating the effect of psychotropic compound activity in triggering physical changes in neural synapses (persistent synapse remodeling) has been generally limited by the availability of adequate experimental technology. A variety of electrical and chemical methods for cell stimulation has been devised over the years. Traditionally, neuronal stimulation is accomplished by electrophysiological techniques such as irreversible stimulation with a metal or glass electrode, either extracellularly or intracellularly. For example, intracellular electrode stimulation, which involves insertion of a glass electrode into a neuron to stimulate and record its electrical activity, incurs a physiological perturbation to the neuron, and is eventually lethal to the cell. This is done acutely, with the cell dying shortly afterwards. Other techniques such as extracellular stimulation with metal or glass electrodes are less invasive, in that they do not actually have to puncture a cell, but can instead be placed in close proximity to the cells to be stimulated. However, extracellular stimulation techniques have limited spatial specificity and will in general stimulate an entire cluster of cells simultaneously and non-selectively, which does not simulate activity in the brain where neurons fire selectively and non-concurrently.

More recently, interfaces between silicon technology and manipulation of living cells have opened new techniques for achieving non-invasive extracellular stimulation. Culturing cells on multi-electrode arrays permits a more spatially specific, yet noninvasive stimulation. When combined with model systems such as dissociated neuronal cultures or organotypic preparations, a silicon interface provides a powerful tool for examining neuronal network behavior (see, e.g., Pine, J., “Recording action potentials from cultured neurons with extracellular microcircuit electrodes,” J. Neurosci. Methods, 2, 19-31, (1980); Gross, G. W., Rhoades, B. K., Azzazy, H. M. E., and Wu, M. C., “The use of neuronal networks on multielectrode arrays as biosensors,” Biosen. Bioelec., 10, 553-567, (1995); Maher, M. P., Pine, J., Wright, J. and Tai, Y. C., “The neurochip: a new multielectrode device for stimulating and recording from cultured neurons,” J. Neurosci. Methods, 87, 45-56, (1999); Kaul, R. A., Syed, N. I., and Fromherz, P., “Neuron-semiconductor chip with chemical synapse between identified neurons” Phys. Rev. Lett., 92, 038102, (2004), all of which are incorporated herein by reference in their entirety). For example, an array of transistor interfaces has been used to stimulate and acquire detailed measurements of membrane potential changes of neurons positioned over a given transistor element. However, the utility of the transistor array is constrained by the fixed spatial position of the transistor elements, e.g., the electrode position, and the grid resolution within an array. In short, the stimulation site is fixed by the electrode position and grid resolution within an array. Therefore the positions of individual neurons in a neuronal network cannot be guaranteed to align perfectly with the transistor elements and thus the individual neurons cannot always be selectively stimulated.

A number of optical methods for eliciting neuronal excitation have also been developed. For instance, cell-specific expression of genetically encoded light-sensitive controllers of membrane voltage can be used to manipulate cell excitability (reviewed in, e.g., Miesenbock, G., Kevrekidis, I. G., “Optical imaging and control of genetically designated neurons in functioning circuits,” Ann. Rev. Neurosci., 28, 533-63, (2005), incorporated herein by reference).

In contrast, photostimulation that triggers neurotransmitter uncaging provides a high degree of experimental flexibility because a small beam of light can be targeted in any region of the sample. Neurons can be incubated either chronically or acutely in solutions containing caged ions, or caged neurotransmitters. These caged ions or neurotransmitters cannot act upon ion channels because they are bound to a carrier molecule that prevents their activity. The carrier molecule can be unbound from the ion or neurotransmitter using a light source such as a laser. Lasers can be used to evoke synaptic responses, for example, by uncaging either the extracellular neurotransmitters or intracellular calcium at nerve terminals to promote synaptic release (see, e.g., Denk, W., Delaney, K. R., Gelperin, A., Kleinfeld, D., Strowbridge, B. W., Tank, D. W., and Yuste, R., “Biological Anatomical and functional imaging of neurons using 2-photon laser scanning microscopy,” J. Neurosci. Methods, 54, 151-162, (1994); Callaway, E. M., and Yuste, R., “Stimulating neurons with light,” Curr. Opin. Neurobiol., 12, 587-592, (2002), both of which are incorporated herein by reference in their entirety). These techniques, although offering the possibility of prolonged stimulation of cells, suffer from the drawback that the cells must be pre-incubated with a special compound that releases the neurotransmitter when excited by a laser. These techniques are therefore invasive. In this type of experiment, however, uncaging complicates interpreting the results of the measurements because the neurotransmitter diffuses away from its normal synaptic localization. For example, uncaged transmitters can activate extrasynaptic neurotransmitter receptors, thereby limiting spatial resolution and complicating data interpretation. Furthermore, there are limitations on the duration of stimulation that is possible.

Recently, a light addressable technique for stimulation of targeted neurons that is based on photoconducting properties of silicon has been developed (see, Colicos, M. A., Collins, B. E., Sailor, M. J., and Goda, Y., “Remodeling of synaptic actin induced by photoconductive stimulation,” Cell, 107, 605-616, (2001), incorporated herein by reference). A light shined on a selected area of a silicon die generates a photocurrent in that area when a voltage is applied to the silicon. By holding the light constant and pulsing the voltage, stimulation of a cell in the selected area can be achieved. This method thereby interfaces a complex neuronal network, such as formed from a group of neurons, with a method that harnesses true random-access capability. In contrast with other techniques described herein which have been mainly used in a research capacity for short-term investigations of basic neuronal function, the photoconducting protocol is unique amongst light-directed cell excitation methods in offering non-invasive stimulation of cells over controlled durations. It is also straightforward to implement and is highly cost-effective. It permits spatially selective excitation of an area within 100 μm², a resolution that is not easily achievable with other methods of extracellular field stimulation.

The photoconductive stimulation protocol has allowed the patterned stimulation of neural networks (X. Y. Tang, R. C. Gerkin, X. L. Wu, Y. Goda, and G. Q. Bi, “Light-directed, patterned stimulation of neuronal networks on silicon chips”, SFN Annual Meeting, Program No. 920.4, (2004)) and the study of activity-dependent synapse formation, by observing the remodeling of synaptic cytoskeleton as a result of stimulation (Colicos, M. A., et al., Cell, 107, 605-616, (2001)). The photoconductive stimulation system has also been modified to integrate a laser beam and an acousto-optic deflector, with which complex spatiotemporal stimulation patterns can be generated to study detailed network properties (Starovoytov, A., Choi, J., Seung, H. S., “Light-directed electrical stimulation of neurons cultured on silicon wafers,” J. Neurophysiol., 93(2), 1090-8, (2005)). In this method, a constant voltage is applied to a silicon substrate, and cells are stimulated using a light source pulsed on to selected areas.

However, hitherto photoconductive stimulation of cells has only been used as a research tool for investigating structural changes that result from long-term cellular excitation, for example, in elucidating how long-term memories are established by neuronal networks. It has not been used to screen compounds for their long-term effects on neuronal networks.

There therefore exists a need in the art for a method that allows the simultaneous testing of the effect of different psychotropic drug compounds on cultured neurons rather than in individual whole animals.

The discussion of the background to the invention herein is included to explain the context of the invention. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY OF THE INVENTION

The invention pertains generally to the field of psychotropic drug discovery. More specifically, the invention pertains to psychotropic drug screening and devices that allow for screening of compounds in an assay format. The invention does not require the use of whole animal models. In one embodiment, the invention includes a method for stimulating neuronal cultures grown on silicon die for extended periods of time such as months, thereby simulating activity that occurs in a mammalian brain. By using photoconductive stimulation and neurons grown on multiple silicon die, the cells can be noninvasively stimulated and maintained for long periods of time. This allows the rapid and cost-effective testing of compounds for their effect on synaptic transmission.

A system for testing psychotropic activity of a compound, the system comprising: a silicon die having a surface suitable for growth of a neuronal network of neurons thereon, and configured to be in contact with a growth medium, wherein the die is immersed in a perfusion medium, and wherein the compound is contained in the growth medium or the perfusion medium; a neuronal network in contact with the surface; a light source configured to direct a light pulse to selectively stimulate the neuronal network for a period of time from about 8 hours to about 1 year; and control circuitry configured to apply a voltage to the silicon die and to operate the light source.

A method for testing psychotropic activity of a compound, the method comprising: growing neurons on a silicon die to provide a neuronal network; stimulating the neuronal network via photoconductive stimulation for a period of time between about 8 hours and about 1 year in the presence of the compound; and performing an electrophysiological test on the neuronal network to determine an effect of the compound.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 0 is a schematic cross-sectional view of a well in one embodiment of the invention;

FIG. 1 shows a long term stimulation device assembly with lid removed;

FIG. 2 shows an exploded view of long term stimulation device assembly;

FIG. 3 shows a side view (some portions shown as cross-sections) of device in both exploded (top) and closed (bottom) forms;

FIG. 4 shows detail of die clamping means (in cross-sectional view);

FIG. 5 shows a light source and control electronics printed circuit board assembly with top and side view;

FIG. 6 shows a base plate printed circuit board interface assembly with top and side views;

FIG. 7 shows a base plate assembly shown in top and cross-sectional view;

FIG. 8 shows a gasket (left), a silicon die (middle), and a pressure applicator (right), in top and side/cross-section views;

FIG. 9 shows a threaded ring (left) and lid (right) in top and cross-section views; and

FIG. 10 shows multiple devices assembled together.

### DETAILED DESCRIPTION

Psychotropic Drugs and Their Action

The central nervous system (CNS) comprises two types of cells: electrically active neurons, and glia which perform a supporting role but are not electrically active. Neurons comprise a central body (soma), a long cylindrical member (the axon) which ends in a terminus or may have branches and thus many termini, and many filamental members (dendrites). The terminus of an axon may be close to the end of a dendrite, forming a structure known as a synapse. Neurons communicate by firing action potentials, which are very rapid changes in the intracellular voltage of the neuron. An action potential is an integration in the soma of electrical signals received from the various dendrites. The action potential is conducted via ion channels away from the cell body through the axon to its terminus where it is converted to a chemical signal, in the form of release of a neurotransmitter. This neurotransmitter crosses the synaptic cleft, the physical space between the axon terminus and the adjacent dendrite, where it binds with receptors on the other side of the synapse, on a different neuron (referred to as the post-synaptic neuron). The released neurotransmitter can be excitatory, in which case it triggers an action potential in the post-synaptic cell, or it can be inhibitory, in which case it would inhibit an action potential in the post-synaptic cell.

Psychotropic drugs can exert effects on the CNS in a number of ways, including but not limited to: affecting neurons presynaptically; acting postsynaptically; and by working on neuronal axons instead of, or in addition to synapses. Mechanistically, the ways psychotropic drugs can work include: preventing the action potential from starting, e.g., by binding to voltage-gated sodium channels, so that no action potential begins even when a generator potential passes threshold; affecting neurotransmitter synthesis, e.g., by increasing synthesis of neurotransmitter precursors such as, but not limited to, L-Dopa, tryptophan, or choline, or by inhibiting synthesis of neurotransmitters such as acetyl choline (ACh); increasing or decreasing the rate of neurotransmitter packaging; increasing or decreasing release of neurotransmitters; acting as agonists by mimicking the original neurotransmitters and activating one or more associated receptors; acting as antagonists by binding to the receptor sites to block activation; preventing neurotransmitter breakdown so that it can act over a longer period of time; and by preventing reuptake. Such effects can manifest themselves in a patient as an increased sensitivity to the five senses, which may arise, for example, from an increased number of signals being sent to the brain. Assessing the influence of drugs that act in any of the foregoing ways is consistent with the practice of the present invention. Furthermore, the activity of the compounds tested relates to any of the processes of a mammalian brain, including, but not limited to: alert function, sleep, and memory formation.

Accordingly, the methods and apparatus of the present invention are applicable to all drug compounds and candidate drug compounds that target brain function, including SSRIs, neuroleptics, antidepressants, antipsychotics, tranquilizers, benzodiazepines, non-phenothiazines, phenothiazines, anti-anxiety drugs, monoamine oxidase (MAO) inhibitors, sedative-hypnotics (non-barbiturate), central nervous system stimulants, anticonvulsants, non-anti-psychotic adrenergics (aromatic, non-catecholamine), as well as anxiolytics and mood stabilizers such as lithium and carbamazepine.

Accordingly, as used herein, the term “psychotropic compound” includes any molecule that is suspected to have psychotropic activity. Such a compound may be a small organic molecule having, say, 50 or fewer non-hydrogen atoms, or a peptide, an oligopeptide, a nucleotide, an oligonucleotide, or a protein, typically a small protein having a molecular weight<5,000 Daltons.

Apparatus

The invention pertains to a screening device for a candidate compound (e.g., a psychotropic drug), wherein the device is designed to replace the use of whole animals in studies of long-term drug effects. The device is also useful in neuroscience applications.

Photoconductive stimulation allows the non-invasive depolarization of neurons cultured on a silicon wafer. The method uses a beam of light to target a cell or cells of interest while applying a voltage bias across the silicon wafer. A targeted cell is excited with minimal physiological manipulation. Although functionally similar to transistor based neuronal interfaces, the photoconductive stimulation method has the advantage of being able to excite any neuron in a network regardless of its spatial position on the silicon substrate. The use of opto/electronics, as opposed to chemicals, as the stimulation system, minimizes the risks of interference by the stimulator on normal cellular activity. Furthermore, using a programmable light-addressed/electric field pulse allows for the stimulation of cells in a pre-defined pattern to simulate specific cellular activity.

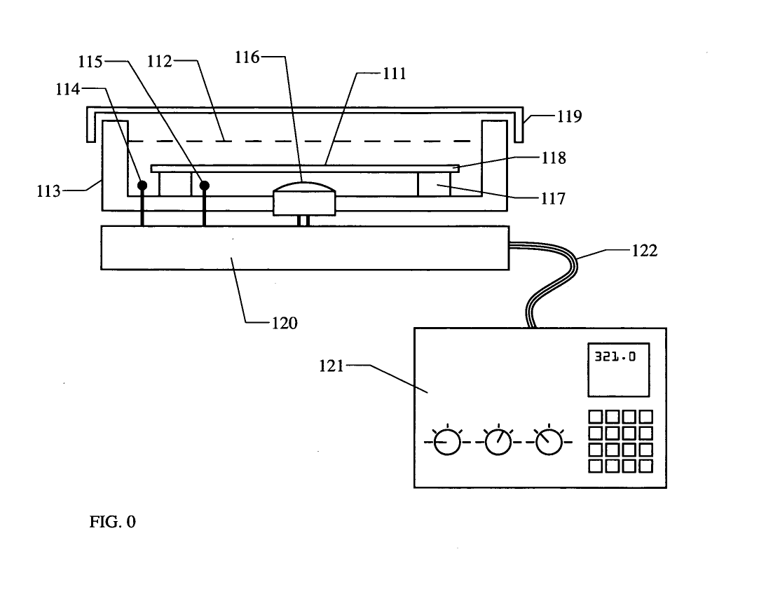

FIG. 0 shows a typical device for use with the present invention, and having the following features: A coated surface 111 of a silicon die 118 upon which cells are grown; an upper level 112 of a fluidic perfusion medium in which the die and, optionally, a candidate psychotropic compound, is immersed; a chamber 113 in which the cells are grown and stimulated; a bath electrode 114, acting as a positive current source to fire the cells; a base electrode 115 acting as a ground; a light source 116, shown targeting a central region of the die; an annular base 117 upon which silicon die 118 is mounted; and a gas exchange lid 119 which covers the chamber and allows CO₂ and O₂ gas to exchange, but retains a sterile environment. It would be understood by one of ordinary skill in the art that other arrangements of the elements shown in FIG. 0 are consistent with the practice of the present invention.

As used herein, the term “light source” means a source of light that can include coherent light, such as a semiconductor laser, or a laser emitting diode, or a dye laser, non-coherent light such as emitted from a light emitting diode. The light beam produced by the light source is preferably incident on a region of silicon surface about 80-120 μm in diameter, and preferably about 100 μm in diameter. It is also consistent with the present invention that the region is 100-500 μm in diameter. Power and light intensity are based upon known parameters for photoconductive stimulation (see Colicos, et al., Cell, 107(5): 605-616, (2001), incorporated herein by reference). A pulse duration of a light source is typically in the range 0.1-10 ms, preferably 0.5-5 ms, even more preferably 1-2 ms, and yet more preferably about 2 ms.

The silicon die 118 modulates the current flow between the electrodes based on the conductivity change that results from being illuminated by the light. Bath electrode 114 and base electrode 115 are preferably made of platinum. A microcontroller 120 is connected to the electrodes, and as further described hereinbelow permits control of the voltage across the die, and the light incident upon it. Microcontroller 120 may be further connected via an interface 122 to an external controller 121. The whole device is self-contained and may be immersed in an incubator. Preferably also, the device is modular and interconnectable so that multiple devices can be joined together and placed easily into an incubator. Preferred arrangements of multiple such devices include 4, 6, 8, 10, 12, 16, and 20 such devices. When multiple devices are connected to one another, they can preferably be controlled by a single external controller 121 Controller 121 as shown in FIG. 0 may have a user interface, such as a keypad, dials and/or a visual display.

As used herein, the term “voltage source” means a source of electromotive force, such as a battery, or an electrolytic cell that is able to supply a voltage of between 3 and 8 V. Any pulse generator capable of producing a 2 ms pulse of 0 to +5 volts can be used to supply the wafer with the desired frequency. Alternatively, more complex stimulation patterns can be achieved by using computer controlled electronics. Logic circuits typically used include those based on TTL, CMOS, and ECL. Transistor-to-transistor (TTL) circuits typically have insufficient power to drive a device of the present invention, and therefore are preferably used only to trigger a power source.

One aspect of the device is its ability to stimulate neuronal cultures for long periods of time. Such periods of time include periods from about 4 hours, to about 1 year, from about 8 hours to about 1 year, from about 1 week to about 1 year, from about 1 month to about 9 months, from about 1 month to about 6 months, from about 2 months to about 4 months, and about 12 hours, about 1 day, about 2 weeks, about 3 months, about 4 months, about 6 months, and about 2 years. It is to be understood, by use of the term ‘about’, that a period is not exact but admits of a small variation. For example, for a period of a month or more, a variation of a small number of days, e.g., 5 days, shorter or longer than the specified period is encompassed. For a period of a few hours, a variation of up to 1 hour more or less than the period is encompassed by use of the term “about”.

By using photoconductive stimulation of neuronal cultures grown on silicon die in a device which can be kept in a cell culture incubator, neurons can be stimulated with activity that both simulates that which occurs in a mammalian brain and can be carried out for long periods of time. At the completion of the stimulation period, the silicon die can be removed from the device and the effect of the prolonged treatment, e.g., on synaptic transmission in the cells, determined by electrophysiological measurements on the cells, as would be familiar to one of ordinary skill in the art, such as, but not limited to: patch clamping, immunochemical staining, treatment with calcium-sensitive dyes, and ultra-structural analysis. Each of the foregoing techniques is useful for giving different types of information on the cell cultures. For example, patch clamping measures responses of single cells; use of calcium sensitive dyes gives information about the connectivity of the network, and permits observation of spontaneous activity patterns; immuno-chemical staining reveals the location of key proteins on the neuronal network; and fixing the neurons, followed by direct observation, such as with electron microscopy, permits study of aspects of the fine structure of the neurons such as the numbers of synapses, their densities, sizes, etc. Important questions that the apparatus of the present invention can assist with answering include questions about the long-term effect of a compound on connectivity changes in a neuronal network, as might be evidenced by a balance between excitatory and inhibitory neurons (that impact physiological conditions such as seizures and cognitive dysfunction). A combination of such techniques can also be applied. For example immuno-chemical staining in conjunction with structural analysis can give rise to a map of receptor distribution around the various neuronal structures. The various electrophysiological techniques can be applied, as previously described, to a silicon die that has been removed from the device. In alternative embodiments it is possible to apply such techniques to the die, while still located in the device.

Silicon die 118 are preferably p-type, 10-20 ohm-cm, boron-doped (1 0 0), single-sided polished, ˜0.5 mm thick. Such die are suitable for opto-electronic stimulation. During operation, each light pulse causes an increase in conductivity of the silicon die below the specific targeted cells by the generation of large numbers of electron-hole pairs liberated by the energy of the light pulse (the photoconductive effect). By applying a voltage pulse across the entire circuit, an electric charge is transferred to the targeted cells resulting in their depolarization. With a rapid sequence of light or voltage pulses, the targeted neurons continually fire as long as the opto-electronic stimulation persists.

In order for the neurons to survive ex vivo for the durations required for prolonged stimulation, such as several months, the die should be kept in a regulated environment. For example, the growth medium and a perfusion medium should have a certain composition that can be controlled, and various other conditions should be kept constant. In particular, the apparatus preferably includes various regulatory systems that ensure that the growth medium and perfusion medium compositions do not deviate significantly from the optimum composition, and to ensure that other conditions remain relatively constant. In particular, the pH of the growth medium should be held at around 7.6±0.1 and can be monitored by using a pH sensor within each device, the media level should always ensure that the die is immersed and can be monitored by a level sensor in each device, the concentration of CO₂ in the apparatus should be kept constant at around 5±0.5, and the temperature should be kept constant in the range 37±1° C. Additionally, the device can comprise a perfusion system for automating changing of the growth media.

It is also preferable that the die is positioned in the device with the aid of a clamping system that not only holds the die in place with minimal motion, but does so in a manner that does not harm the cells on the surface of the die, for example because only the top edges of the die surface are in contact with the die, and which also permits ready removal of the die.

In an exemplary embodiment of this invention, the silicon die are first coated with growth substrate, consisting of a poly-d-lysine pre-coat, laminin, and in some cases Matri-Gel (Sigma). Cells are cultured onto the die using standard cell culture techniques. The growth medium may be formulated according to many that are known in the art for culturing neurons, and preferably consists of a mixture of BME (Basal Medium Eagle), D-Glucose, Glutamine, HEPES, Na-Pyruvate, FBS, Pen-Strep, and B27, in proportions as further described in the examples herein. Compounds to be tested are then added to individual die. The compounds can be contained in the growth medium, or can be regulated at desired levels by including them in the perfusion medium. Although it is preferable that the present invention analyses the effects of a single compound on a given neuronal network, it is consistent with the methods herein that several psychotropic compounds could be added simultaneously to a given neuronal network. It is also consistent that a compound could be delivered in conjunction with a caging agent.

The light source for targeting the region of the die to be stimulated is provided by a semiconductor laser (or alternately, a light emitting diode (LED)). Although this is shown in FIG. 0 disposed underneath the die, embodiments of the invention can also have the light source situated above the die. For the duration of the assay, the light source is activated to target a sub-region of the die; the timed pulses of electric stimulation can be generated by applying a voltage waveform between the two electrodes. The frequency and pattern of stimulation can be completely controlled through an external controller 121 connected via an interface 122 such as a wire, or wirelessly, to the microcontroller 120 attached to the base of the well plate. The frequency and pattern of stimulation, which constitutes the process, can be designed to stimulate neuronal activity observed in a mammalian brain.

Also included in the device is a microprocessor 121 that communicates with a microcontroller 120 which has programmable control of the light source and the electric field applied across the silicon die. The microcontroller 120 has firmware that enables the timed, scheduled application of various activity patterns which simulate activity observed in an intact brain. The microcontroller 120 also has control over the intensity of both the light and electrical pulse, in order to adjust these factors for the successful firing of the neurons on the silicon die. Such adjustments may apply to just the light source, just the voltage, or both in combination, and are within the capability of one of ordinary skill in the art.

The voltage and light sources are configured so that a variety of patterns of stimulation can be applied to a neuronal network. In particular, the voltage and light sources are preferably initially calibrated to ascertain specific voltages and light intensities that are able to fire at least a proportion of the neurons in the neuronal network. For example, calibration might be based on a conductance of the circuit that includes the silicon die. It is also consistent with the practice of the present invention that the voltage and light sources are calibrated during the period under which stimulation is occurring. For example, during a 3 month stimulation, it is consistent that a frequent recalibration is carried out, such as weekly, or daily, to take account of changes in conductance of the solution.

Exemplary stimulation protocols can be devised to probe different types of neuronal behavior, corresponding to, for example, sleep or waking. It is not necessary, and not desirable to apply constant stimulation. Thus, preferred protocols are based on pulses of light of durations in the range 2-5 ms at a time, pulsed at a frequency of, say, 10 Hz, and applied at intervals of once per hour, or once per half hour. In an alternate approach, a 2 ms pulse may be applied at a frequency of 1 Hz continuously. Voltage ranges are typicall in the range of 5-10 V

It is also preferable to apply the voltage and light pulses concurrently and simultaneously.

In another embodiment, the invention provides a system in an assay format suitable for testing psychotropic drug candidates in high throughput screening. Such a system is comprised of: an assay plate comprising at least one location, each location having a surface suitable for mounting a removable silicon die and maintaining an environment for cell growth; an optical/electronic targeting and firing system; and a microprocessor to control the interface.

The assay plate may be plastic or other suitable material. Each location in the assay plate may be a well in the assay plate. Each location comprises a mounting block for a removable silicon die and is filled with growth media. Each well contains electrodes such as platinum electrodes for the stimulation of the neurons. Each location may contain a separate light source for targeting a subset of neurons grown on the silicon die.

In some embodiments, each well comprises a direct wired, or wireless, connection from an external controller to electronic circuitry embedded in the wells. The electronics control the light intensity, pulse intensity, pulse frequency, and the pattern of pulse application.

One embodiment of the apparatus of the present invention is suitable for working with multiple dies, all of which are incubated in a single chamber. Such an embodiment can consist of a sterile chamber inside which is a plate containing multiple wells. Within each well, a removable silicon die is mounted. Within each well, a light source is mounted underneath or above the silicon die. Electrodes are placed in growth media above and below the silicon die. Primary neuronal cultures are then grown on the silicon die by placing neuron cells in each well of the device. A specific cellular adhesion substrate is preferably coated onto the silicon die to promote cellular growth and survival. The sterile chamber is maintained in an incubator for a period as previously described herein, but preferably as long as several months, and is controlled by the microprocessor, thereby inducing brain-like activity in the neuronal network. This stimulation can be carried out in the presence of a test compound, such as a psychotropic drug candidate. At the completion of the stimulation period, the die can be removed and subjected to electrophysiological and biochemical testing to determine the effect of the test compound on the neuronal network.

A further embodiment of the present invention uses multiple instances of a single die contained in a chamber, thereby requiring multiple chambers. Each device consists of a sterile chamber that contains a removable silicon die upon which neuronal cultures are grown, in a growth medium. Multiple chambers are included in a single device. A user-defined region of the silicon die is targeted by a light source. Electrodes both underneath and above the silicon die provide an electrical pulse which fires the targeted neurons. The device defines the dynamics required in terms of the light source and electrical pulse to provide noninvasive long-term stimulation of the neurons. The removable silicon die containing a neuronal culture is maintained in a controlled environment in each chamber for long periods of time (e.g., up to six months) in the presence of the test compound(s) under investigation. The device also includes an interface through which programmed patterned activity is scheduled.

A further embodiment consists of a sterile device which can hold and fire several neuronal cultures grown on silicon die simultaneously. The device can consist of a number of different wells depending on the application (e.g., a single well, 6 wells, or 12 wells), each containing a removable die of silicon. Platinum electrodes above and below each die provide a circuit when the well is filled with liquid media. Below or above the silicon die, a light source such as a semiconductor light source is used to target a specific sub-region of the silicon die carrying the culture. The array of wells is designed to be functional under conditions appropriate for cellular growth, and to remain functional for long periods of time.

Processes

The present invention includes a method for determining the effect of candidate compounds (e.g., psychotropic drugs) on neurons.

A method of the present invention generally includes placing neurons on a substrate coating the silicon die. The die is then placed in an incubator to support growth. During the stimulation, changes in the neuronal connectivity and synaptic biochemistry occur. A test compound can be added to the neuronal network on the die, and its effect on these processes will be manifested in the resulting network. Once removed and studied by standard electrophysiological and biochemical methods, the neuronal networks on the plates provide data comparable to a whole animal model.

A process of using the device described herein includes the preparation of the material of the silicon die, which is chosen for its conductivity, crystal structure, surface preparation, and thickness. A process of using the device also includes treatment of the silicon surface to support the growth of the neuronal cultures, as well as the methods needed for long-term maintenance of these cultures. Electronics incorporated in the device allow control over the light and electrical pulse firing sequence.

The invention also provides a method for simulating brain activity for extended periods in cultured neurons, the method comprising: growing or plating neurons on a silicon die in an apparatus as described above; stimulating neuronal network activity for extended periods, of between about 8 hours and about a year, via photoconductive stimulation; and removing the silicon die and still living neurons, and performing an electrophysiological test on them, as can be carried out with a whole animal tissue sample. It is to be understood that the method also comprises running a control in parallel with tests on the effects of a compound. In such a control, a neuronal netowork is cultured, placed in an apparatus as described, and stimulated for the same length of time, but no compound is present.

The invention also includes a method for the study of the long-term effects of such compounds on neuronal connectivity and function by using cultured neurons grown in an incubator on silicon die. Using this technique and multiple devices, multiple test compounds can be simultaneously screened with an in vitro system and their effect on synaptic transmission determined. By eliminating the need for animal testing during the screening process for psychotropic drugs, not only can new drugs be identified much more rapidly, but also it can be done in a much more humane and inexpensive manner. Not only can drugs be identified that modify synaptic function for psychotropic purposes, but also the consequences and side effects of these drugs can also be studied.

### EXAMPLES

### Example 1

### Exemplary Assay Device

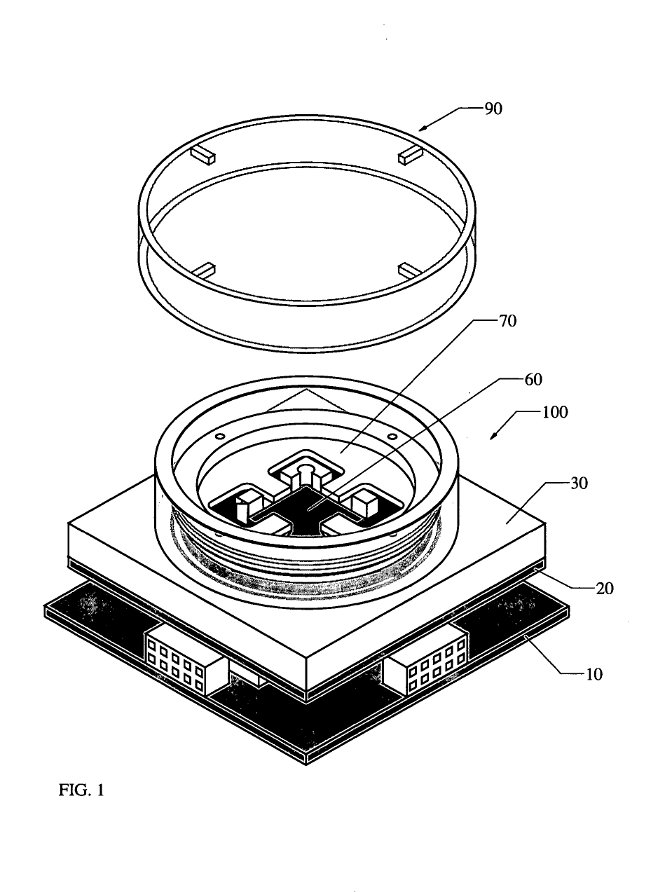

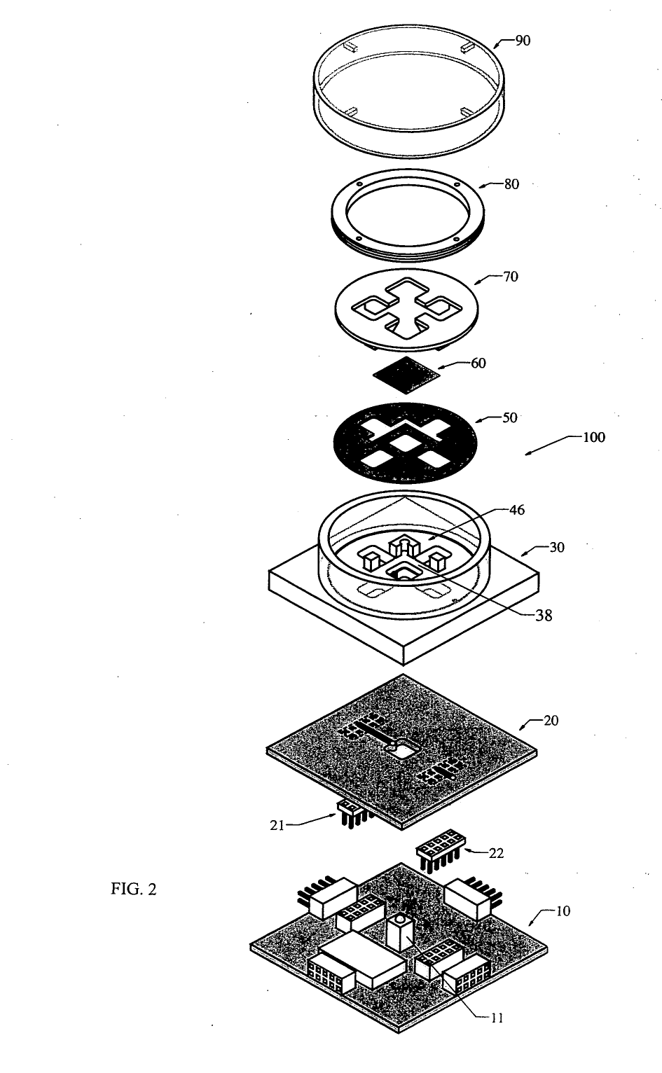

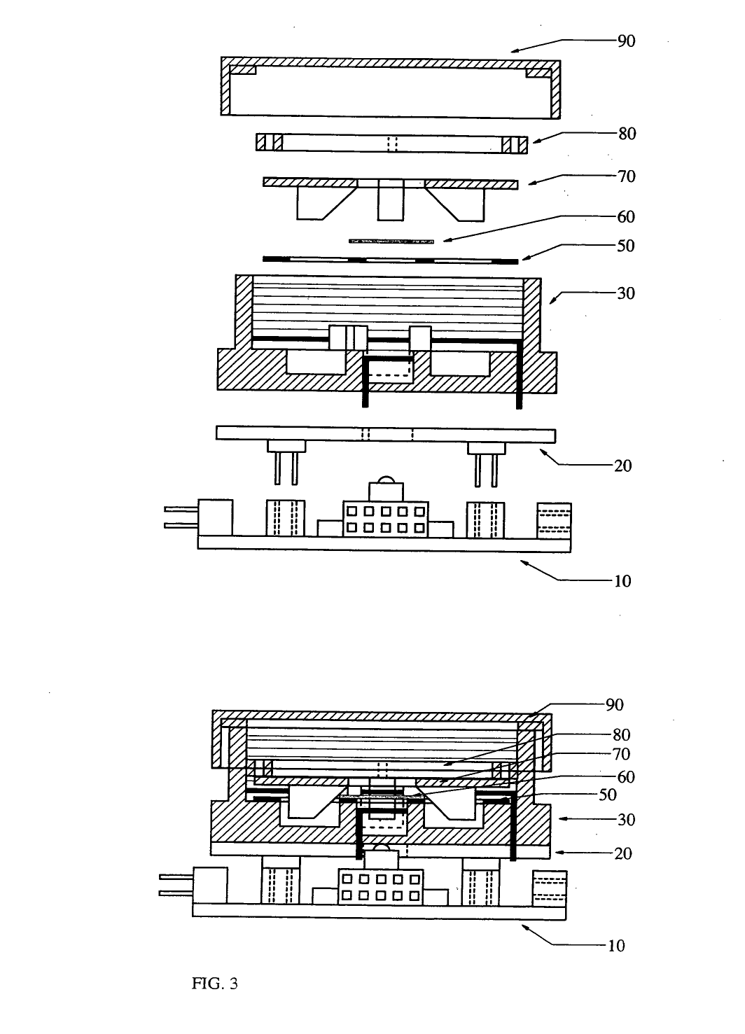

One embodiment of the long term stimulation device 100 is shown in perspective view in FIG. 1, with lid 90 removed. The device 100 is comprised of a number of subassemblies, which are shown in exploded view in FIG. 2, and in cross-section in FIG. 3.

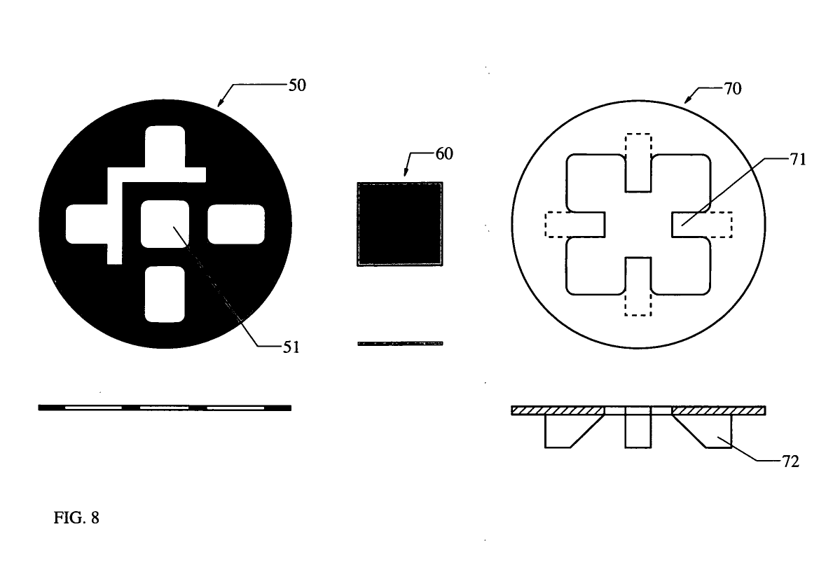

Base unit 30 has a well 46 which houses the silicon die 60. Silicon die 60 is sandwiched between a silicone rubber gasket 50, underneath, and a pressure applicator 70 on top. For proper operation of photoconductive stimulation, the top and bottom surfaces of the silicon die 60 must be kept electrically isolated from each other once the die is placed into the base unit 30. To achieve this, a silicone rubber gasket 50 is placed between the surface 38 of the primary well 46 of the base unit 30 and the silicon die 60. The silicon die 60 is slightly oversized compared to the dimensions of an aperture 51 in the middle of gasket 50 (see also FIG. 8). This ensures that a portion of the gasket 50 will be present around the outer perimeter of the bottom surface of the die 60. Downward pressure is applied to the silicon die by the pressure applicator 70 to enable a fluid tight seal to be formed around the outer edge of the bottom surface of the die 60. Tightening or loosening the threaded ring 80 controls the amount of pressure that is applied to the applicator 70. Interface printed circuit board 20 is mounted to the bottom surface of base plate 30. Two header pin assemblies 21 and 22 are mounted on the bottom of the interface board 20 to provide a detachable means of connecting to the controller printed circuit board 10. A light source 11 is mounted on the controller board 10. A separate interface board 20 is used to allow the base unit assembly (30 and 20) to be easily separated from the controller board 10 for cleaning and maintenance.

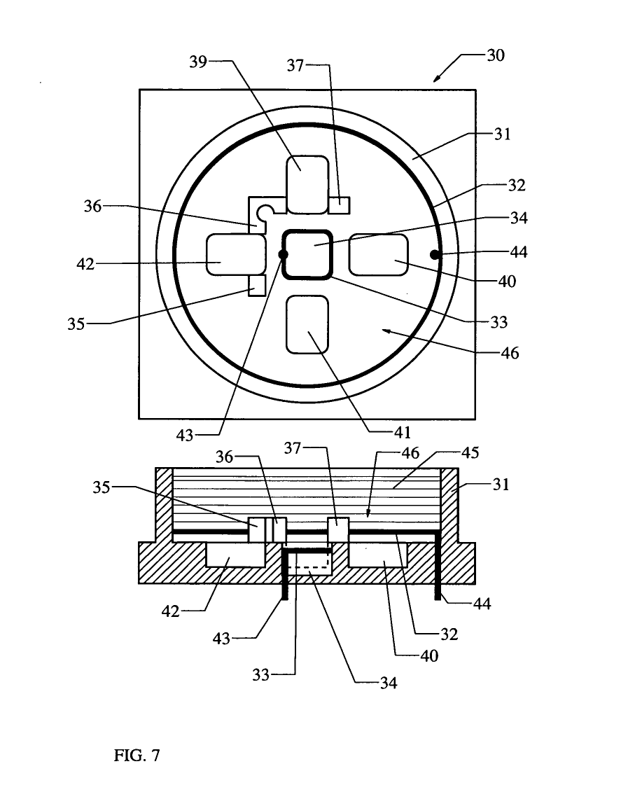

FIG. 7 shows the base unit 30 in further detail. The base plate 30 is made of an optically transparent material. During the operation of the device, the well 46 (and the secondary bottom well 34, which contains the bottom electrode 33) of the base unit will contain a growth medium (fluid) that provides the environment necessary for the survival and growth of the cells. This solution is electrically conductive. Within the main well 46 and bottom well 34 of the base unit are two platinum electrodes 32 and 33 which, in conjunction with the conductive media, provide electrical contacts on the top and bottom surface, respectively, of silicon die 60 (not shown). The electrodes can be made from platinum wire having 0.5 mm diameter, and 99.9% purity (with Iridium), (as may be obtained from, e.g., Sigma-Aldrich, Cat# 267228). The silicon die 60 is slightly oversized compared to the dimensions of the bottom well 34. The inner sidewalls 31 of the main well 46 are threaded 45 to accept the threaded ring 80. Alignment blocks (35, 36, 37) are provided to ease the task of placing the die 60 in the correct location. The base plate 30 has four wells (39, 40, 41, 42) into which wedges 72 (not shown) on the under-surface of applicator 70 fit.

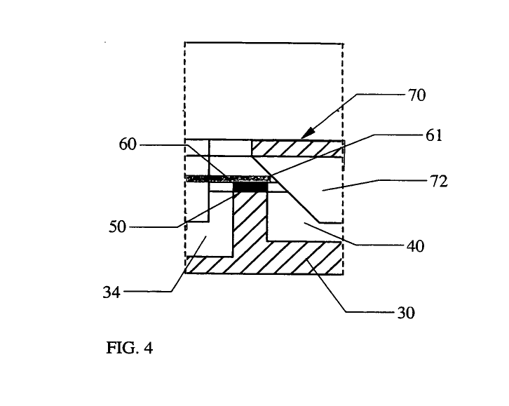

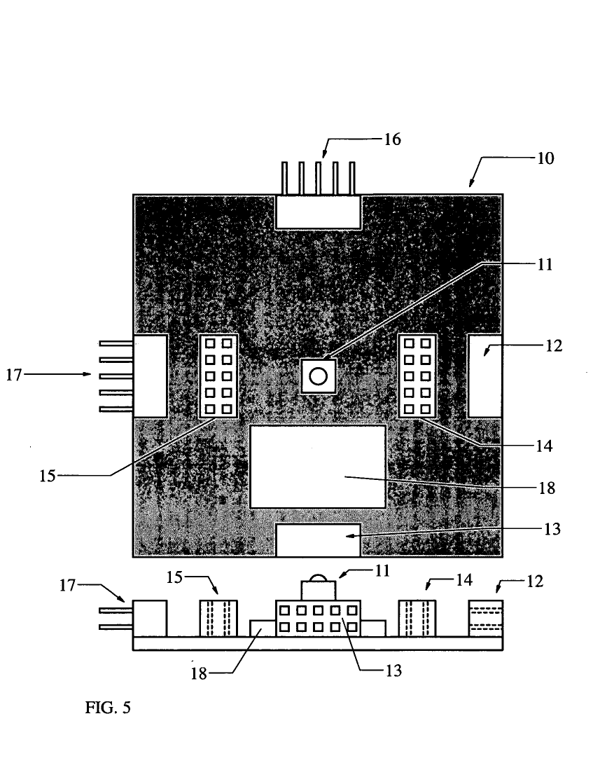

FIG. 4 shows detail of structures that clamp the die in place. FIG. 8 shows pressure applicator 70 in top and cross-sectional view. To minimize harm to the cells growing on the top surface of silicon die 60, the pressure applicator 70 uses a series of wedge shaped protrusions 72 on its bottom surface to minimize the area of the applicator 70 which is in contact with die 60. As shown in FIG. 5, the applicator's wedges 72 contact only the upper edge 61 of die 60. Equal pressure is applied to all four sides of the die 60, to ensure a fluid tight seal around the entire perimeter of the bottom surface of the die 60.

The pressure applicator 70 is constructed of a plastic material so that individual fingers 71 that come into contact with the die 60 have some flexibility in the vertical direction. This helps ensure that the pressure applied to all sides of the die 60 is approximately equal even if small irregularities exist in the surface 38 of the base plate 30, the gasket 50, the die 60, or the applicator 70 itself. The applicator's wedges 72 fit into four wells (39, 40, 41, 42) on base plate 30. There is sufficient clearance between these four wells and the wedges 72 to ensure that small variations in die 60 size and thickness can be accommodated.

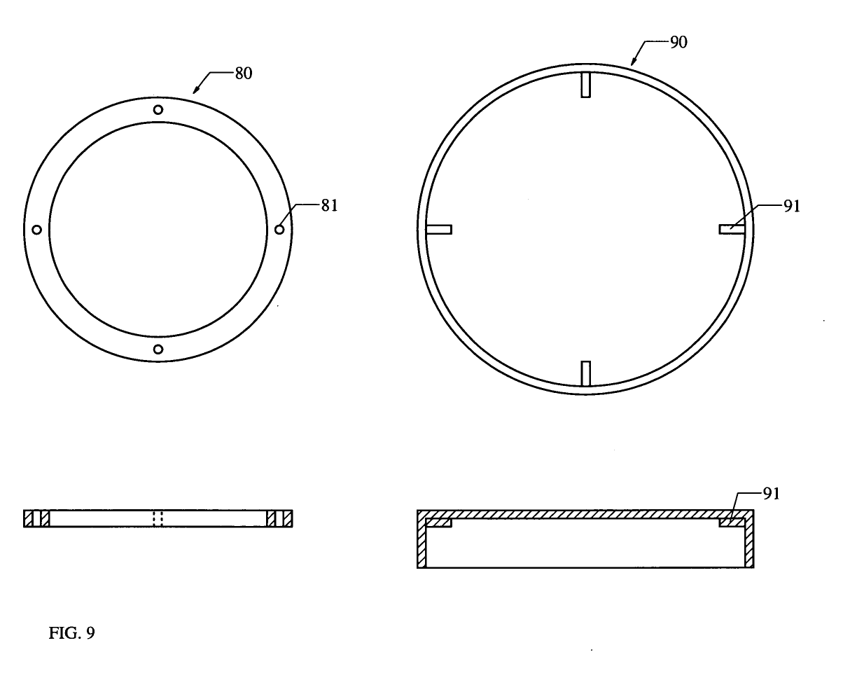

The threaded ring (FIG. 9) has small holes 81 into which a tool can be inserted to allow the ring to be easily turned.

The device cover 90 (FIG. 9) has four small protrusions 91 on its inner surface so as to provide a small air gap between the cover and the base plate 30. This allows CO₂ and O₂ gases to exchange between the interior and the exterior of the device, while retaining a sterile environment.

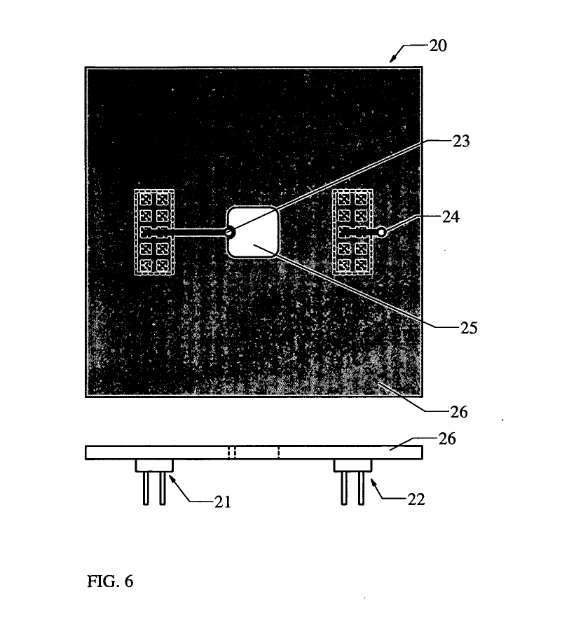

The base plate 30 is mounted on the interface printed circuit board 20. The interface board 20 is shown further in FIG. 6. Board 20 has an opening 25 that is aligned with the location of the bottom well 34 of the base plate 30. The top 32 and bottom 33 electrodes (not shown) are fed through the bottom of the base plate 30 by short stubs (44 and 43 respectively) which connect to points 24 and 23 of the interface printed circuit board 20 that is mounted on the bottom surface of the base plate 30. The header pin assemblies 21 and 22 on the interface board 20 along with their corresponding header sockets 15 and 14 on the controller board 10 provide both an electrical connection to the bottom 33 and top 32 electrodes as well as structural support for the base plate 30 and interface board 20 assembly. In this manner the base plate assembly 30 and board 20 can be easily removed from the controller board 10 for cleaning and other tasks.

As shown in FIG. 5, the light source 11, mounted on the controller printed circuit board 10 situated below the interface board 20, will shine upwards through the opening 25 of the interface board 20 (see FIG. 6) and through the optically transparent bottom surface of the bottom well 34 in the base plate and illuminate the central region of the die 60. The region of the die 60 that is illuminated will become conductive (through the photoconductive effect, by which incident photons generate electron-hole pairs within the silicon thereby increasing the conductivity in the volume that is illuminated). A voltage source is connected between the top 32 and bottom electrodes 33. Normally, the un-illuminated die has sufficiently low conductivity that the top 32 and bottom electrodes 33 are electrically isolated and no significant current flows between the electrodes. However, when the die 60 is illuminated with sufficient strength, a conductive channel will form between the top and bottom surfaces of the die 60 at the site of the illumination.

The surfaces of the die 60 have a very thin layer of silicon dioxide grown on them that has a high resistance to current flow. Therefore, even though a conductive channel exists within the die's volume, the silicon dioxide on the top and bottom surfaces will prevent a significant steady-state (DC) current from flowing between the top 32 and bottom 33 electrodes. At both the top and bottom surfaces of the die, in the illuminated region, there is a sandwich of conductive fluid (the growth media), insulator (silicon dioxide), and conductive silicon (due to the illumination). This combination of material constitutes a capacitor, thereby allowing a current (AC current or displacement current) to flow across the die if the applied voltage is time varying. Therefore, when the die is illuminated and a voltage pulse (or any time dependent waveform) is applied across the electrodes, a current will flow through the illuminated region. If the current is of sufficient strength, it will depolarize cells that are sitting on the die 60 immediately above the illuminated region, thereby “firing” the cell(s). Although in this embodiment, the light source is turned on prior to activating the voltage pulse, an alternate approach is to use a light pulse in combination with a constant voltage across the electrodes to induce the “firing” current. Similarly, both the light and voltage sources can be time dependent.

As shown in FIG. 5, the controller board 10 contains the light source 11 used to induce the photoconductive region in the die 60, and a controller module 18 used to provide local control of the light source as well as generating the required voltage signal to be applied across the electrodes. The control module 18 is typically comprised of a number of components which could include (not specifically shown): a micro-controller, a digital to analog converter, one or more amplifiers, an analog to digital converter, and a voltage regulator, among others. The controller module 18 also provides an interface to an external device controller that is used to setup an individual stimulation unit 100. The controller 18 controls characteristics such as: light source strength and on/off timing; voltage pulse amplitude, shape, duration, and repetition rate. These factors can be adjusted to ensure successful firing of the cells on the die 60. The controller 18 can be programmed to provide the timed, scheduled application of various activity (firing) patterns which simulate activity observed in the intact brain. The controller board 10 is also provided with edge connectors 12, 13, 16, 17, allowing multiple units to be structurally connected together, as well as providing a common electrical interconnection bus between all units.

The light source assembly 11 may comprise a semiconductor laser. Alternatively, a light emitting diode (LED) may be used. Optics may be required to allow the light provided by the source to be focused to the appropriate spot size on the surface of the die. The required intensity of the light will depend on factors such as light's wavelength, optical transmission characteristics of the base plate 30 and the growth media, as well as thickness of the die. Therefore, the appropriate settings for the light source are best determined empirically in conjunction with determining the electrical characteristics of the voltage pulse source.

In an alternate embodiment of the long term stimulation device, the light source 11 can be incorporated into the device cover 90 so as to illuminate the die 60 from the top.

To facilitate survival of the cells during the long term stimulation experiments, a pH sensor can be embedded into the main well 46 of the base plate 30. Over time, cell respiration causes change in the pH of the media in the main well 46. If the media's pH is allowed to deviate significantly from the norm, cell death will occur. Therefore, the pH sensor can be used to monitor the media pH and alert the operator that a media change is required. Alternately, a perfusion system can be attached to each well that provides for a slow but continuous change of the media. In this case, the pH sensor can be used to adjust the flow rate of a CO₂ bubbler embedded within the external media tank of the perfusion system, thereby allowing the pH of the media to be altered.

A level sensor can also be incorporated into the main well 46 of the base plate 30 to provide a means of monitoring the level of the growth media in the well. If the fluid level drops below the desired level, the control system can alert the operator that additional media must be added. Alternately, if a perfusion system is incorporated into the overall system, the level sensor can be used to adjust the media flow rate to ensure that the desired depth of media is maintained at all time in the main well 46.

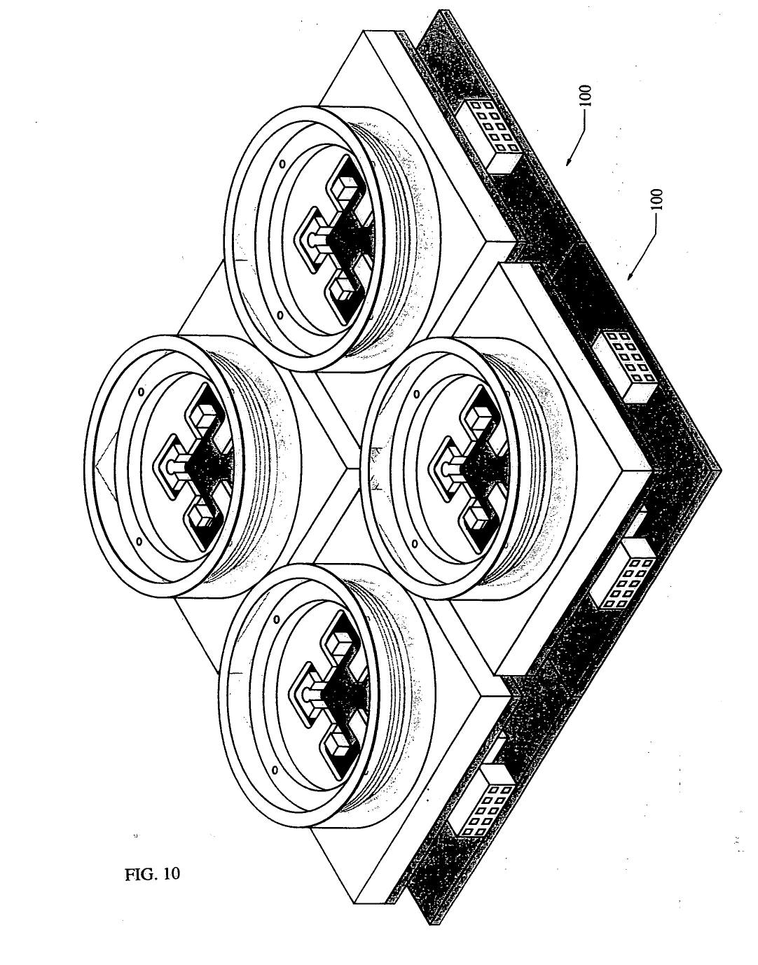

Each device 100 is configured so that multiple instances can be connected together thereby allowing multiple tests to be run in parallel. An example of an array 200 of four devices, configured in a 2×2 array, is shown in FIG. 10. Accordingly device 100 can be used singly, or in arrays of various dimensions. Each controller module associated with a device 100 may have a means of uniquely identifying an individual device 100, so that each unit within an array 200 can be programmed to use a different test procedure. A single external controller can be plugged into one of the edge connects 12, 13, 16, or 17 associated with a device 100 and used to program the individual units 100 within array 200.

In the exemplary embodiment shown in the accompanying figures, the silicon die 60 is first coated with growth substrate, consisting of a poly-d-lysine pre-coat, laminin and in some cases Matri-Gel (Sigma). The cells of interest are cultured on the silicon die 60 using standard cell culture techniques and then placed into the well 46 of base unit 30 for long term stimulation experiments. Compounds to be tested are then added to individual wells. The silicon die 60 is obtained by dicing (cutting) a silicon die which has the following characteristics: p-type, 10-20 ohm-cm, boron-doped (1 0 0), single-side polished, and approximately 0.5 mm thick. Each die is approximately 10 mm by 10 mm in size. A thin layer (preferably 2 nm or less) of silicon dioxide is grown on the surface of the die prior to use. This can be done by exposing the die to an ozone rich environment for several minutes at room temperature, followed by storing in ethanol. An acceptable layer can also be grown by heating the die in air.

### Example 2

### Culturing Neurons

Reagents

The following reagents were used.

Cell culture: Standard procedures for preparing dissociated neuronal/glial co-cultures on glass slides can be followed until the point of plating. Rodent hippocampal neurons taken from newborn pups can be used routinely. The following solutions are used to produce high viability co-cultures of medium neuronal density (5×10⁴ cells per chip).

Dissection solution: a solution was made up having the following composition: Hank's Balanced Salt Solution (10× stock, Gibco, Cat#14185-052) 100 ml; HEPES (Sigma, Cat# H-7523) 2.4 g; pH adjusted to 7.2 with NaOH (1N); topped up with water to 1,000 ml; osmolarity adjusted to 310 mOsm with Sorbitol (˜0.182 g/mOsm/l). The solution was filtered and sterilized into 2×500 ml bottles using a 0.2 μm filter, and stored at 4° C.

A growth medium is formed by mixing the following ingredients: BME (Basal Medium Eagle, Gibco, Cat#21010-046) 42.5 ml; D-Glucose 0.3 g; Glutamine (Sigma, Cat#G-8540) 500 μl (200 mM stock frozen into 500 μl aliquots at −20° C.); HEPES (1M, Sigma, Cat# H-7523) 500 μl (1M stock in 500 μl aliquots at −20° C.); Na-Pyruvate (100 mM), Gibco, Cat#11360-070 500 μl; FBS (Standard, Hyclone Lot#KPD20979, Cat#SH30397.03) 2.5 ml (aliquoted and stored at −20° C.); Pen-Strep (Gibco, Cat# 15070-063) 1 ml (aliquots stored at −20° C.); and B27 (Gibco, Cat# 17504-044) 1 ml (aliquots stored at −20° C.).

Enzymatic buffer: CaCl₂ (150 mM)+L-Cysteine (100 mM): 30 μl; dissection solution (as above): 2 mls; EDTA (50 mM): 20 μl; Worthington Papain: 80 μl (available from Worthington Biochemical Company, Cat# LS003126). The mixture is heated to 37° C. for 30 min., and filtered sterile with 0.2 μm filter.

Silicon Die Preparation

The following materials and reagents were used.
Borate buffer: Boric acid (MW: 61.83, 1.24 g), and Sodium tetraborate (MW: 381.37, 1.90 g) were added to 400 mls of H₂O, and brought to pH to 8.5 with NaOH.
Poly-D-Lysine: 1 mg/ml stock in H₂O (Sigma, Cat# P7280, 5 mg).
Laminin: 1 mg/ml stock (Sigma, Cat #L2020, laminin, 1 mg/ml).
Silicon die: 385-400 μm thickness, (1 0 0) crystal lattice orientation, 10 ohm-cm resistivity (Silicon Quest, 1230 Memorex Drive, Santa Clara, Calif. 95050-2812).

The procedure is as follows:

1 cm-sided silicon dies are cut from die, 15-20 cm in diameter, carefully cleaned with a swab soaked in an ethanol solution, as applicable. A layer of silicon oxide is formed on the polished surface of the silicon die. The die are then sterilized with concentrated ethanol.

Aliquots of ˜400 μl of a working concentration of 1 μg/mL poly-D-lysine diluted in borate buffer were added to each well, and the die were allowed to sit at room temperature overnight. Laminin is then applied at 1 μg/mL, diluted from stocks in PBS around 3-4 hours prior to cell plating. The laminin is then thoroughly washed from both sides of the die prior to plating the cells.

Cell Culture Preparation

The following is a brief description of a technique for preparing rat hippocampal cultures. Further description can also be found in Colicos, M. A., et al., “Remodeling of synaptic actin induced by photoconductive stimulation,” Cell, 107, 605-616, (2001), incorporated herein by reference. Tissue processing: Hippocampi from 8 Sprague Dawley newborn pups (sufficient for 48 die) were removed and placed on ice in dissection solution. Hippocampi were then chopped using the tip of a glass Pasteur pipette, breaking each hippocampus into approximately 4 pieces, to allow better enzyme access. Hippocampi were then transferred with minimal residual dissection solution to pre-warmed, filtered protease solution and incubated for 30 minutes at 37° C. in a CO₂ incubator, with the top of the tube loosened.

Cell trituration: 2 standard glass pipettes were heated to reduce the size of the tip opening to ⅓ and ¼ of the original diameter. A third pipette was heated to remove the sharp edge from the tip without substantially altering the bore size. Following incubation in the protease mix, the hippocampi were washed 3 times in warm media using the polished pipette, and then re-suspended in 1 ml of media. Cells were then triturated successively through the reduced bore pipettes, passing the full cell volume each time, approximately 10 times, first with a larger tip diameter pipette followed by a smaller diameter tip pipette.

Plating: cell suspension density was determined by counting on a hemocytometer, and then diluted in media to achieve the desired plating density for the appropriate number of die. We routinely use 5×10⁴ cells per 1 cm² die. Cultures were grown in the incubator undisturbed for 3 days, at which time the media was refreshed by removing one half the volume and replacing it with fresh warmed media.

The foregoing description is intended to illustrate various aspects of the present invention. It is not intended that the examples presented herein limit the scope of the present invention. The invention now being fully described, it will be apparent to one of ordinary skill in the art that many changes and modifications can be made thereto without departing from the spirit or scope of the appended claims.

What is claimed:

1. A system for testing psychotropic activity of a compound, the system comprising:
a silicon die having a surface suitable for growth of a neuronal network of neurons thereon, and configured to be in contact with a growth medium, wherein the die is immersed in a perfusion medium, and wherein the compound is contained in the growth medium or the perfusion medium;
a neuronal network in contact with the surface;
a light source configured to direct a light pulse to selectively stimulate the neuronal network for a period of time from about 8 hours to about 1 year; and
control circuitry configured to apply a voltage to the silicon die and to operate the light source.

2. The system of claim 1 wherein the silicon die is located in a well on a plate.

3. The system of claim 1, further contained in an incubator.

4. The system of claim 1 further comprising one or more sensors.

5. The system of claim 4, wherein the one or more sensors includes a pH meter.

6. The system of claim 1, further configured to regulate one or more of pH, temperature, partial pressure of CO₂, and level of perfusion medium.

7. The system of claim 1, wherein the control circuitry comprises a first electrode, a second electrode, a voltage source, and a microcontroller.

8. The system of claim 1 wherein the light source is configured to direct the light pulse in a defined pattern at a defined wavelength.

9. The system of claim 7 wherein the voltage source is configured to direct a voltage pulse in the surface of the silicon die.

10. The system of claim 7, wherein the voltage source is configured to provide a voltage sufficient to fire one or more of the neurons in the neuronal network.

11. The system of claim 1, further comprising an incubator.

12. The system of claim 1, wherein the period of time is about 3 months.

13. The system of claim 1, wherein the period of time is about 6 months.

14. An array of systems according to claim 1, wherein each of the systems is located in a well of an assay plate.

15. An array of systems according to claim 11, wherein each system of the array of systems is independently controllable.

16. A method for testing psychotropic activity of a compound, the method comprising:
growing neurons on a silicon die to provide a neuronal network;
stimulating the neuronal network via photoconductive stimulation for a period of time between about 8 hours and about 1 year in the presence of the compound; and
performing an electrophysiological test on the neuronal network to determine an effect of the compound.

17. The method of claim 16 wherein the silicon die is placed in a regulated environment to support growth.

18. The method of claim 16 wherein the photoconductive stimulation is controlled by a microcomputer.

19. The method of claim 16, carried out in an assay format.
